(19) United States
(12) Patent Application Publication
(10) Pub. No.: US 2011/0189050 A1
(43) Pub. Date: Aug. 4, 2011

(54) PROBE SYSTEM FOR MEASURING A VARIABLE OF A MEDIUM IN A CONTAINER, ESPECIALLY FOR STERILE APPLICATIONS

(75) Inventors: Rainer Schlereth, Neuss (DE); Joachim Oppermann, Gemmrigheim (DE); Hermann Straub, Rottenburg (DE)

(73) Assignee: Endress + Hauser Conducta Gesellschaft fur Mess- und Regeltechnik mbH + Co., KG, Gerlingen (DE)

(21) Appl. No.: 12/984,632

(22) Filed: Jan. 5, 2011

(30) Foreign Application Priority Data
Jan. 29, 2010 (DE) DE102010 001391.9

(51) Int. Cl. A61L 2/18; G01N 1/00

(52) U.S. Cl. 422/28; 73/64.56

(57) ABSTRACT

A probe system for measuring a measured variable of a measured medium, especially a measured liquid, contained in a process container; comprises:

a probe housing, which is connectable to the process container by means of a process connection; an immersion tube axially shiftable between a measuring position, when extended out of the probe housing, and a treatment position, when retracted into the probe housing, wherein a measuring probe having a measuring head is securable in the immersion tube, wherein, in the treatment position, the measuring head is arranged within a treatment chamber formed in the probe housing; and a piston, which is arranged in an axially displaceable manner in a cylinder adjoining the treatment chamber on a side of the treatment chamber away from the process connection, and which is connected with the immersion tube, wherein a fluid line serving as a supply line for a sterilization medium opens into the cylinder on a side of the piston facing the process connection.

The invention relates to a probe system for measuring a measured variable of a measured medium in a process container, especially for sterile applications.

The field of probe systems for measuring physical or chemical, measured variables of a medium—e.g. of a fluid, especially a liquid—in process technology is diverse. For example, the manufacture of sterile solutions in pharmaceutical or food technical processes requires the use of measuring probes for monitoring the product and the process, respectively. Such measuring probes include pH measuring probes, conductivity measuring probes, or optical or electrochemical measuring probes for determining concentration of a substance contained in the medium to be monitored, e.g. O₂, CO₂, certain ion types, or organic compounds.

In the state of the art, it is known to perform inline-measuring of fluids, e.g. products of chemical processes, in the case of which probe systems are put to use, which include an axially moveable immersion tube, in which a measuring probe is held. Such probe systems are referred to as ‘retractable assemblies’. These retractable assemblies are secured on a process container, for example a pipe conveying the fluid to be monitored. These retractable assemblies include a treatment chamber, into which, during operation, the measuring probe can be temporarily moved by means of the immersion tube for calibration or washing/rinsing purposes, to be therein brought into contact with a calibration liquid, and/or a washing or rinsing medium for cleaning. When the calibration or washing/rinsing procedure is finished, the measuring probe can thereafter be moved back into the fluid to be monitored, in order to continue with the inline-measuring. In such case, contamination of the fluid to be monitored with calibration liquid, or, conversely, a contamination of the calibration liquid with the fluid to be monitored, is opposed with the assistance of seals, which seal the treatment chamber and the process container from one another.

For retractable assemblies for use in sterile processes, an additional, second treatment chamber has been proposed. Thus, in DE 38 20 405 C2, for example, a retractable assembly is described, which has a housing having a housing nozzle connectable to a reaction vessel, wherein, in the housing, an immersion tube with a measured value sensing probe, whose measuring head is surrounded by a perforated cage, can be moved back and forth, from a measuring position beyond the housing nozzle into a retracted, rest position in the housing. As a drive for the immersion tube movement, the retractable assembly includes a cylinder, in which a piston connected with the immersion tube is manually, pneumatically or hydraulically shiftable between two end positions. In the rest position, the opening of the housing nozzle is outwardly sealed by means of a closure. In the rest position, the front part of the immersion tube facing the reaction vessel, with the measuring head of the probe, is located in a first rinsing chamber of the housing. The first rinsing chamber has an inlet and an outlet. Neighboring the first rinsing chamber in the housing is a second rinsing chamber. The second rinsing chamber is located farther away from the reaction vessel and has its own inlet and outlet. In the rest position, a rinsing and/or sterilizing of the measuring probe and parts of the immersion tube can be performed, by sending rinsing or sterilization medium through the first and the second rinsing chambers. In this way, there can also be cleaned and sterilized those parts of the immersion tube and/or the probe, which, in the case of extending the probe out into the measuring position, enter into the first rinsing chamber. It should therewith be prevented that parts of the immersion tube and the probe not washed or rinsed and sterilized enter into the rinsing chamber, in which, in given cases, medium from the reactor vessel is contained, such that the medium becomes contaminated. Through the provision of a second rinsing chamber, the constructed height of the retractable assembly as compared to conventional retractable assemblies with only one rinsing chamber increases by at least the of height of the second rinsing chamber. At the same time, due to the two rinsing chambers arranged one after the other, a certain minimum length for the measuring probe is required, in order that this can be held in the immersion tube and be electrically contacted.

Also described in the German patent application DE 10 2005 051 279 A1 is a retractable assembly for use in sterile processes. It includes a housing, which has a space for accommodating a measuring probe, and a first chamber for accommodating a first fluid—e.g. a calibration fluid or a washing or rinsing fluid. The first chamber is connected with the space for accommodating the probe. The housing furthermore includes a connecting section, by means of which the housing is connectable or connected with a container, wherein the container serves for accommodating a medium, whose property is to be measured with the probe. In the housing is arranged between the first chamber and the connecting section of the housing at least one second chamber for accommodating a second fluid (such as, for instance, sterile water), which should behave uncritically in comparison to the first fluid as well as also in comparison with the medium. In the case of defective seals, the probability of a contamination of the medium by the first fluid as well as a contamination of the first fluid by the medium should then be lessened.

The probe is accommodated in an immersion tube, in which openings are provided, which give access for the medium or a rinsing liquid to the measuring head. The probe can be moved axially within the housing between a measuring position, in which the probe, with its measuring head including a sensor, protrudes into the medium; and a washing or rinsing position, in which the measuring head is arranged inside of the first rinsing chamber. For this, the retractable assembly, like the retractable assembly described in DE 38 20 405 C2, includes a piston, which is movably seated in a cylinder and connected with the immersion tube, and which, pneumatically driven, is axially movable between two end positions. In order also to assure in the washing or rinsing position a sealing off of the second rinsing chamber arranged between the first rinsing chamber and the medium from the medium, the distance between the front end of the probe and the openings in the region of the measuring head of the probe must be larger than the axial length of the second rinsing chamber—i.e. than the distance between two seals—which seal off the second rinsing chamber from the medium, on the one hand, and, on the other hand, from the first rinsing chamber. As a result, this effects that, for a certain immersion depth of the measuring head in the medium in measurement operation, a stroke of the immersion tube must be produced, which is enlarged by the axial length of the second rinsing chamber. Additionally, the front end of the immersion tube reaching beyond the position of the actual measuring head by exactly this length requires a corresponding amount of space, so that the retractable assembly can only be used on process containers with a relatively large minimum volume.

It is, therefore, an object of the present invention to provide a probe system, especially a retractable assembly, which is suitable for application in sterile processes, and overcomes the disadvantages of the state of the art. The probe system should especially be constructed compactly and in a space saving manner.

This object is achieved by a probe system for measuring a measured variable of a measured medium, especially a measured liquid, contained in a process container, comprising:
A probe housing, which is connectable to the process container by means of a process connection;
an immersion tube axially shiftable between a measuring position, when extended out of the probe housing, and a treatment position, when retracted into the probe housing, wherein a measuring probe having a measuring head is held in the immersion tube, wherein, in the treatment position, the measuring head is arranged within a treatment chamber formed in the probe housing;
a piston, which is arranged in an axially displaceable manner in a cylinder adjoining the treatment chamber on a side of the treatment chamber away from the process connection, and which is connected with the immersion tube, characterized in that a fluid line serving as a supply line for a sterilization medium opens into the cylinder on a side of the piston facing the process connection.

The cylinder is preferably formed by the probe housing. Since the cylinder is arranged neighboring the treatment chamber on a side of the treatment chamber facing away from the process connection, in the treatment position of the immersion tube, a section of the immersion tube is located within the cylinder on the process connection facing side of the piston, which, in the measuring position of the immersion tube, is arranged at least partially within the treatment chamber. This section of the immersion tube can be sterilized by supplying sterilization medium to the cylinder via the fluid line, while the immersion tube is located in the treatment position. In this way, an entrance of germs from the cylinder into the treatment chamber during the moving of the immersion tube into the measuring position is prevented.

A comparable sterilization effect is thus achieved as in the case of the retractable assembly known from DE 38 20 405 C2; however, the length of the probe housing can be reduced by the length of the second sterilization chamber required in the known retractable assembly. This also permits the use of shorter measuring probes. At the same time, material is saved, which also reduces the manufacturing costs of the probe system.

The axial shifting of the piston and, respectively, of the immersion tube can occur manually, or by means of a pneumatic, electrical or hydraulic drive.

The pneumatic drive is formed by the cylinder and the piston displaceably seated therein and connected with the immersion tube. In this embodiment, the piston divides the cylinder into a first pressure chamber on the side of the piston facing the process connection and a second pressure chamber on a side facing away from the process connection, wherein the fluid line serving as a supply line for a sterilization medium opens into the first pressure chamber, and wherein a fluid line serving as a supply line and/or as a discharge line for a pressurizing medium opens into the second pressure chamber.

Since a fluid line opening into the first pressure chamber for supplying a sterilization medium is provided in the first pressure chamber, besides its function as a pressure chamber for the pneumatic or hydraulic drive of the axial movement of the immersion tube, the first pressure chamber can be utilized as a sterilization chamber, in which at least one subsection of the immersion tube can be sterilized. A sterilization medium fed via this fluid line, e.g. superheated steam, can simultaneously be used as a pressurizing medium for actuating the drive. If superheated steam is thus introduced under pressure into the first pressure chamber, so that a higher pressure occurs in the first pressure chamber than in the second pressure chamber, the piston is shifted axially in the direction away from the process connection. This shifting effects an axial shifting of the immersion tube connected with the piston into the treatment position. At the same time, a sterilizing of the first pressure chamber and of the section of the immersion tube located therein occurs through the sterilization medium, e.g. the superheated steam.

In addition to the fluid line serving as a supply line for a sterilization medium, the probe system can have another fluid line, which opens into the first pressure chamber, and which serves as a supply line and/or as a discharge line for a pressurizing medium into and out of the first pressure chamber.

In this case, in an operating method for the probe system, pressurizing medium is first introduced via the first supply line opening into the pressure chamber, in order to shift the piston in the direction away from the process connection. This shifting effects an axial shifting of the immersion tube connected with the piston into the treatment position. After reaching the treatment position, sterilization medium for sterilizing the first pressure chamber and the section of the immersion tube arranged therein is introduced via the additional fluid line serving as a supply line for the sterilization medium and opening into the first pressure chamber.

When the pneumatic drive is a linear drive, the section of the immersion tube accommodated in the pressure chamber in the treatment position is exactly as long as the section arranged in the first treatment chamber. This has the result that the entire length of the immersion tube arranged within the treatment chamber in the measuring position can be sterilized. In this way, a transporting of germs from the region of the retractable assembly facing away from the process into the treatment chamber during the shifting of the immersion tube into the measuring position is prevented.

The immersion tube can have on its front end—which is immersible in the fluid—a protective cylinder, wherein the measuring head of the measuring probe is arranged within a perforated region of the protective cylinder.

Advantageously, the treatment chamber and the cylinder are arranged directly neighboring one another. This can, for example, be implemented by forming, within the sensor housing, between the treatment chamber and the cylinder, an axial guide for the immersion tube, wherein the guide includes a seal, which seals off the treatment chamber and the cylinder from one another.

The probe system can, besides the (especially additional) fluid line opening into in the first pressure chamber, which serves as a supply line for a sterilization medium in the first pressure chamber, include another fluid line opening into in the first pressure chamber, which serves as a discharge line for the sterilization medium from the first pressure chamber. Other supply and/or discharge lines for different cleaning and sterilization media for the first pressure chamber can be provided.

Preferably, at least one of the fluid lines opening into the first pressure chamber is closable by means of a valve.

The invention relates furthermore to an operating method for a probe system for measuring a measured variable of a measured medium contained in a process container,

wherein the probe system has a probe housing, which is connectable to the process container by means of a process connection,
and wherein the probe system has an immersion tube axially shiftable between a measuring position, when extended from the probe housing, and a treatment position, when retracted into the probe housing, wherein a measuring probe having a measuring head is held in the immersion tube, wherein, in the treatment position, the measuring head is arranged within a treatment chamber formed in the probe housing,
and wherein the probe system furthermore has a piston, which is arranged in an axially displaceable manner in a cylinder adjoining the treatment chamber on a side of the treatment chamber away from the process connection, and which is connected with the immersion tube, wherein, via a fluid line serving as a supply line for a sterilization medium opening into the cylinder on a side of the piston facing the process connection, a sterilization medium is introduced into the cylinder.

In a first step of the method, the immersion tube can be shifted into the treatment position, and, in the treatment position of the immersion tube, in a second step, a sterilization medium can be introduced into the treatment chamber, and a sterilization medium can be introduced into the cylinder via the fluid line opening into the cylinder, and, in a third step, the immersion tube can be shifted into the measuring position.

The introducing of sterilization medium into the treatment chamber and into the cylinder can selectively occur simultaneously or one after the other.

The invention will now be explained in greater detail on the basis of the appended drawing, the figures of which show as follows:

FIG. 1 a schematic axial sectional drawing of a probe system with an immersion tube in the measuring position;

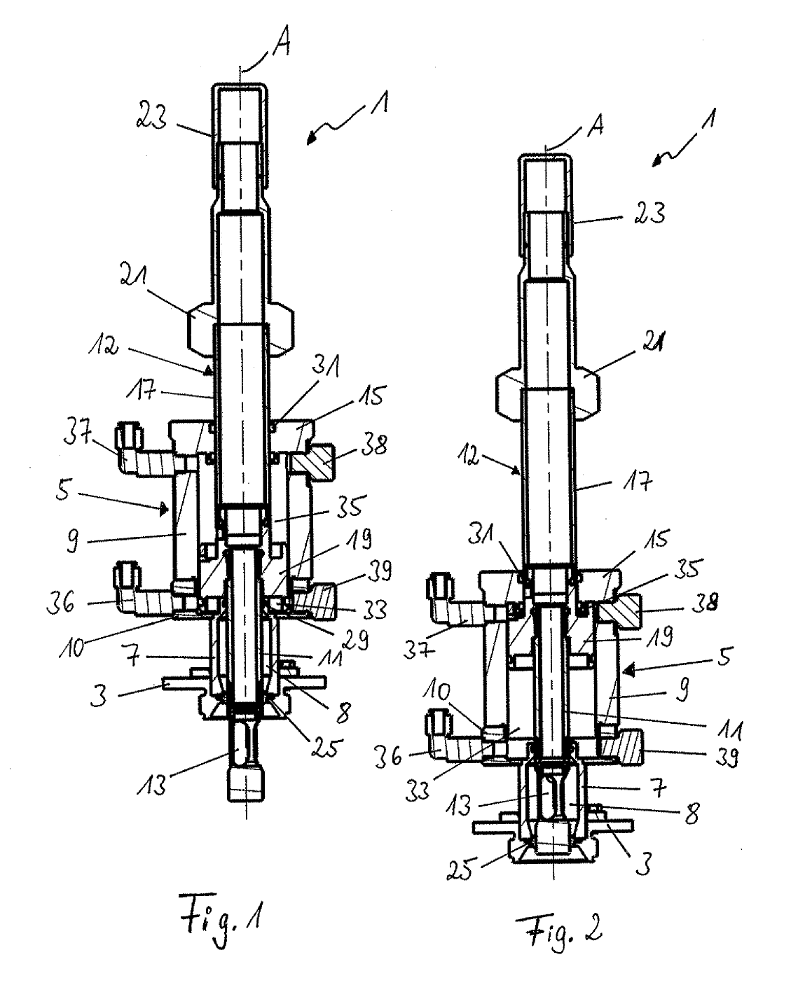

FIG. 2 a schematic axial sectional drawing of the probe system illustrated in FIG. 1 with the immersion tube in the treatment position;

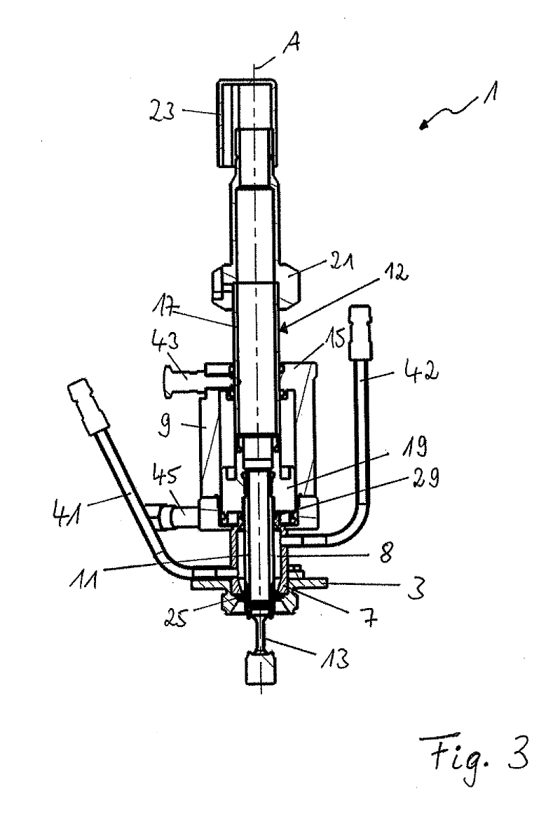

FIG. 3 a further schematic axial sectional drawing of the probe system, in which supply lines to the treatment chamber are shown;

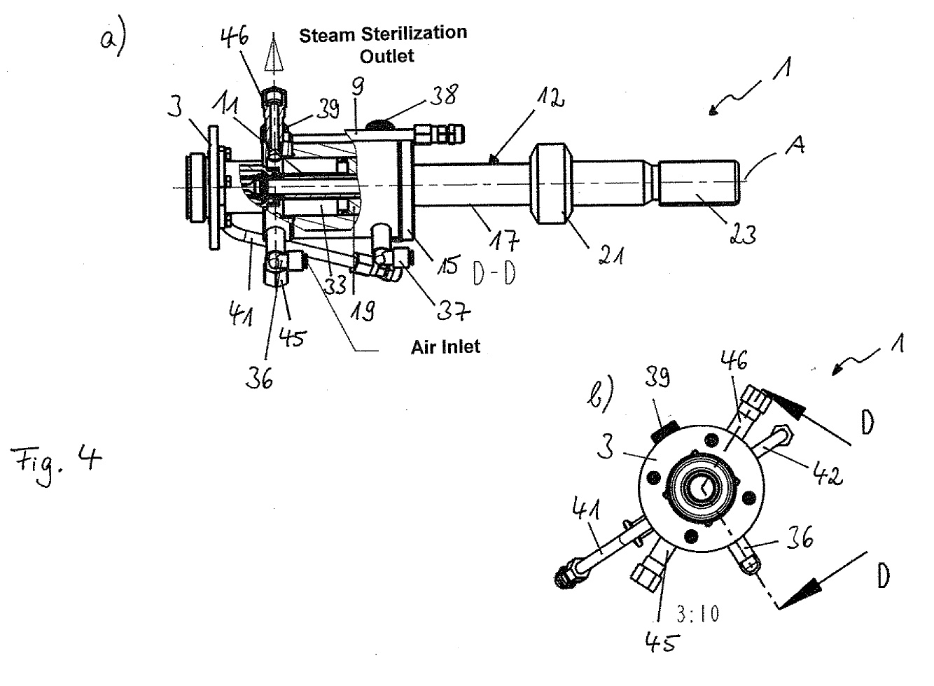

FIG. 4 a further schematic axial sectional drawing of the probe system along the cutting planes D-D, in which, on the one hand, a supply line for a pressurizing medium and, on the other hand, a discharge line for a sterilization medium are to be seen;

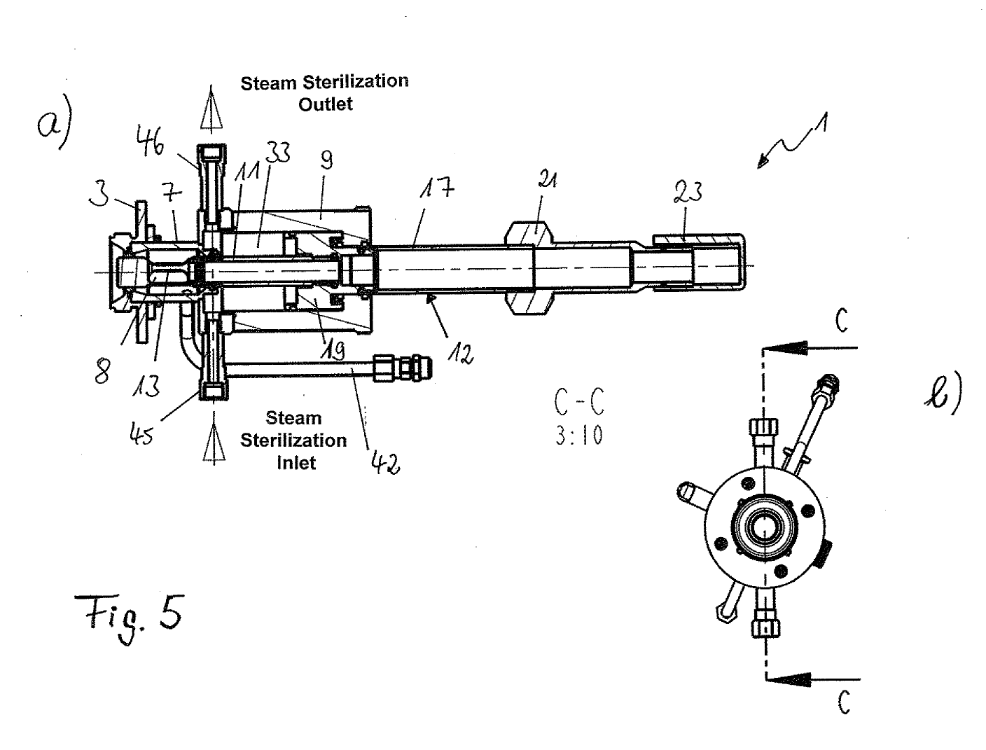

FIG. 5 a further schematic axial sectional drawing of the probe system along the cutting plane C-C, in which the supply and discharge lines for sterilization medium into and from the cylinder are visible;

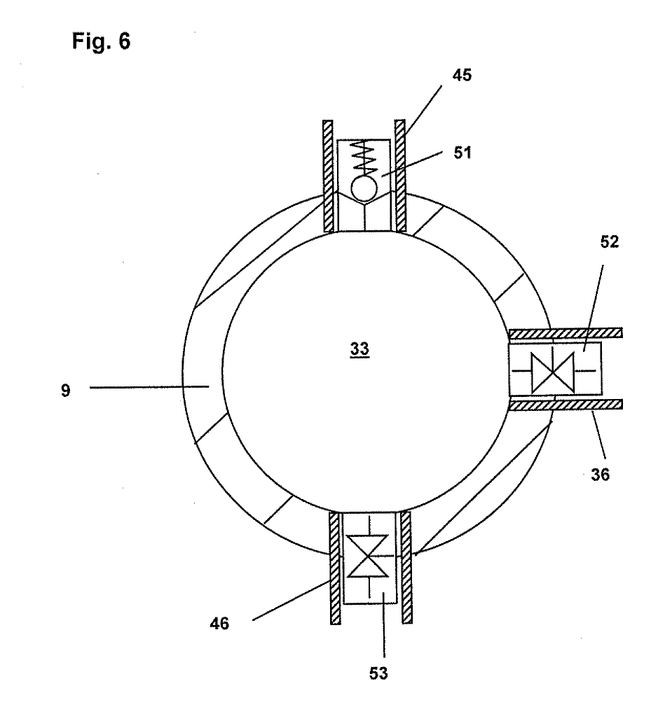

FIG. 6 a schematic cross sectional representation of the cylinder.

FIG. 1 shows a schematic axial sectional drawing of a probe system 1 in measuring position. FIG. 2 shows the same probe system 1 in an axial sectional drawing in treatment position. Probe system 1 possesses a probe housing 5, which, by means of a process connection 3, can be affixed to a process container (not shown). The process container can be, for example, a pipeline, through which a process medium, especially a liquid, flows. The process connection 3 is, in the example of FIGS. 1 and 2, embodied as a connection part with a flange, which can be connected by means of screws to a complementary flange of the process container. Alternatively, the process connection 3 can also be embodied as a milk tube adapter, as an APV connection or as a Varivent connection.

Probe housing 5 is, in the example illustrated here, embodied in the form of a plurality of parts. It especially includes a treatment chamber part 7 connected with the connection part forming the process connection 3, and a cylinder 9, arranged neighboring the treatment chamber part 7 on the side of the treatment chamber part facing away from the process connection. The treatment chamber part 7 and the cylinder 9 are both secured to a connector 10, and sealed off from one another as well as from the environment. The treatment chamber part 7 and the cylinder 9 can also be constructed in other embodiments in the form of a plurality of parts.

Guided axially displaceably in the probe housing 5, i.e. along the central axis A of the probe housing 5, is a stroke tube 12, which is embodied as a plurality of parts. The central axis A coincides with the cylindrical symmetry axis of the stroke tube 5, the cylindrical symmetry axis of the process connection 3, the cylindrical symmetry axis of the treatment chamber part 7 and the cylindrical symmetry axis of the cylinder 9. The stroke tube 12 includes an immersion tube 11, which serves for accommodating and holding a measuring probe (not shown) with a measuring head. For performing measurements, the measuring head of the measuring probe is brought in contact with the measured medium, for example a process liquid in the process container. The measuring probe can be, for example, a pH single-rod measuring chain, which has a measuring head including a pH-sensitive, glass membrane. Such a measuring probe is also referred to as a pH, glass electrode. The measuring probe can be accommodated and secured in the immersion tube 11 in such a way, that the measuring head is arranged within a perforated region 13 formed on the front end of the immersion tube 11. The front end with the perforated region 13 thus forms a protective cylinder for the measuring head. Such a measurement probe arrangement in an immersion tube is known, for example, from DE 20 2007 017 297 U1. In the measuring position of the immersion tube 11 (FIG. 1), the immersion tube 11 with the perforated region 13 protrudes into the process vessel containing the liquid medium to be examined, so that the perforated region 13 is supplied, especially flowed through, with medium. In this way, between the measuring head of the measuring probe accommodated in the immersion tube 11 and the medium, a contact sufficient for performing measurements, e.g. of the pH-value of the medium, is assured. In the treatment position of the immersion tube 11 (FIG. 2), the perforated region 13 is arranged within the treatment chamber 8 formed by the treatment chamber part 7.

The stroke tube 12 furthermore includes a guide tube 17 connected with the immersion tube 11. The stroke tube 12 furthermore includes on its end region facing away from the process connection a grip part 21, as well as a protective cap 23. The cylinder 9 is closed on its end facing away from the process connection 3 by a floor 15, which possesses a central opening serving as a guide for an axial shifting movement of the guide tube 17.

The guide tube 17 is secured together with the immersion tube 11 to a piston 19, for example, by a screwed connection or by means of a bayonet connection. Piston 19 is, in order to match the inner space of the cylinder 9, embodied cylindrically symmetrically with respect to the central axis A, and is arranged so as to slide within the cylinder 9 in such a manner that it divides this into a first, process connection side, pressure chamber 33 and a second pressure chamber 35 on the side away from the process connection. By supplying the first or second pressure chambers with a pressurizing medium, the piston 19 is shiftable in the direction, and counter to the direction, of extension of the immersion tube 11 in the axial direction, i.e. along the central axis A. For supplying and discharging pressurizing medium into/from the first pressure chamber 33, there serves a fluid line 36 opening into in the first pressure chamber 33. For supplying and discharging pressurizing medium into/from the second pressure chamber 35, there serves a fluid line 37 opening into the second pressure chamber 35. In this way, a pneumatic drive for the axial movement of the immersion tube 11 is formed. Equally, a shifting of the piston 19 can be achieved by producing a negative pressure in one of the pressure chambers 33, 35. Optionally, end switches 38, 39 can be provided, which register the reaching of the respective end positions of the piston 19 on the process-side stop formed by the connector 10 or on the rear-side stop formed by the floor 15 and, in given cases, forward a report to a control device (not shown).

The connection part forming the process connection 3 includes an internally lying seal 25, which, both in the measuring position (FIG. 1) as well as in the treatment position (FIG. 2) of the immersion tube 11, seals off the treatment chamber 8 from the process container. In the measuring position, the seal 25 lies against the immersion tube 11 on the side of the perforated region 13 facing away from the process container. In the treatment position, the seal 25 lies against the immersion tube 11 on the side of the perforated region facing the process container. The seal 25 can be composed, for example, of a sealing ring arranged in an encircling annular groove of the connection part.

The treatment chamber 8 is sealed from the cylinder 9 by at least one annular seal 29 sealing an annular gap between the end of the treatment chamber part 7 facing away from the process connection 3 and the immersion tube 11. On its end facing away from the process connection 3, the cylinder 9 is sealed off from the environment by a seal 31 sealing the annular gap between the floor 15 and the guide tube 17.

FIG. 3 shows an axial sectional drawing of the probe system 1 illustrated in FIG. 1 and FIG. 2 from another perspective. In this representation, opening into the treatment chamber 8 are to be seen a first fluid line 41 serving for supplying, and a second fluid line 42 for discharging, washing, rinsing or calibration liquid. In the treatment position (FIG. 2), washing, rinsing, cleaning and calibration media, especially washing, rinsing, cleaning and calibration liquids, can be supplied via these fluid lines 41, 42. For sterile applications, a sterilization medium, e.g. superheated steam, can, moreover, be supplied via these fluid lines into the treatment chamber 8 and then discharged, in order to sterilize the treatment chamber 8, the region of the immersion tube 11 arranged in the treatment chamber 8, the seal 29, and the region of the measuring probe—especially the measuring head—exposed by the perforated region 13 to the sterilization medium.

In the axial sectional drawing of FIG. 3, a locking bolt 43 is furthermore visible, which can be used for securing the stroke tube 12, for example, for maintenance purposes, e.g. for the replacement of the measuring probe accommodated in the immersion tube 11.

Shown in the axial sectional drawing of FIG. 3, opening into the cylinder 9, especially into the first pressure chamber 33 of the cylinder 9, is furthermore a fluid line 45, which serves for supplying sterilization medium into the first pressure chamber 33. Here, there are thus provided two separated fluid lines 36, 45 for supplying pressurizing medium and sterilization medium. In an alternative embodiment, a single fluid line can also be provided for delivery, on the one hand, of pressurizing medium into the first pressure chamber, and, on the other hand, of sterilization medium into this pressure chamber 33. In a variant, sterilization medium, especially superheated steam, can be fed under pressure into the first pressure chamber 33, in order to actuate the pneumatic drive. At the same time, the pressure chamber 33 is, in this way, sterilized during the shifting of the piston 19.

In FIG. 4 a) there is presented an axial section through the probe system 1 taken along the cutting planes D-D, as shown in the plan view of the probe system 1 illustrated in FIG. 4 b). FIG. 5 a) shows an additional axial section through the same probe system 1 along the cutting plane C-C, as shown in the plan view of the probe system 1 illustrated in FIG. 5 b). In both representations, the immersion tube 11 is located in the treatment position, i.e. the immersion tube 11 is retracted into the probe housing 5 and the perforated region 13 of the immersion tube 11 is located within the treatment chamber 8. In the case in which a measuring probe is accommodated in the immersion tube 11, the measuring head of the measuring probe arranged within the perforated region 13 can be supplied with a medium fed into the treatment chamber 8 via the supply line 41, e.g. in order to clean or to sterilize the measuring head or in order to perform a calibrating of the measuring probe.

In the representations of the probe system 1 illustrated in FIG. 4 and FIG. 5, an additional first fluid line 45 opening into the first pressure chamber 33 and an additional second fluid line 46 opening into the first pressure chamber 33 are to be noticed. The first additional fluid line 45 serves for supplying sterilization medium, e.g. superheated steam, into the first pressure chamber 33. The second additional fluid line 46 serves for discharging the sterilization medium from the first pressure chamber 33.

The interplay between the fluid line 36 opening into the first pressure chamber 33 for delivery of pressurizing medium and the two additional fluid lines 45 and 46 opening into the first pressure chamber 33 for supplying or discharging sterilization medium can be controlled by means of valves which can open or block the fluid lines in one or more directions. This is illustrated in the schematic cross sectional view of the cylinder 9 shown in FIG. 6. For example, the supply line for the sterilization medium formed by the fluid line 45 can be sealed from the pressure chamber 33 by means of a check valve 51 blocking the flow of medium from the pressure chamber 33. The discharge line for the sterilization medium formed by the second additional fluid line 46 and the fluid line 36 serving alternately as a supply line and as a discharge line for pressurizing medium of the pneumatic drive for the shifting movement of the stroke tube 12, and therewith also of the immersion tube 11, can, in each case be selectively opened or closed to the pressure chamber by means of a shut-off valve 52, 53, especially pneumatically actuatable shut-off valves. The shut-off-valves 52, 53, which also are referred to as “on/off valves”, can be embodied in many different ways. For example, they can be embodied as normally open valves, i.e. in such a manner that they are opened in their unactuated “rest position”, and can be closed electrically, pneumatically or hydraulically. Alternatively, the shut-off valves 52, 53 can be embodied as normally closed valves, i.e. embodied in such a manner, that they are closed in their unactuated rest position, and can be opened electrically, pneumatically or hydraulically. Of course, the shut-off valves 52, 53 can also be mechanically actuatable, especially by hand. In the following, the function of the shut-off valves 52, 53, is described, for example, on the basis of the variant with pneumatically closable shut-off valves opened in rest position.

Should the immersion tube 11, for purposes of cleaning, calibrating and/or sterilizing the measuring probe accommodated therein, be retracted into the probe body 5, a pressurizing medium, e.g. pressurized air, is introduced into the first pressure chamber 33 via the fluid line 36 for supplying pressurizing medium and through the normally open, shut-off valve 52. At the same time, the pressurizing medium can be utilized to close the shut-off valve 53 of the additional second fluid line 46, which serves for discharging sterilization medium. The additional first fluid line 45, which serves for supplying sterilization medium, is sealed off by the check valve 51 from the first pressure chamber 33. Thus, within the first pressure chamber 33, increased pressure in comparison to the second pressure chamber 35 can be built up, which effects an axial movement of the piston 19 in the direction away from the process connection 3.

If the immersion tube 11 has reached the treatment position, a sterilization medium, which the check valve 51 allows to pass, can be fed via the first additional fluid line 45 into the pressure chamber 33. At the same time, the shut-off valve 52 of the fluid line 36 for delivery of pressurizing medium into the first pressure chamber 33 is pneumatically closed, while the shut-off valve 53 of the second additional fluid line 46 for discharging sterilization medium is opened. This can, for example, be effected by making use of the sterilization medium, in that, for example, sterilization medium is fed via a T-fitting from the first additional fluid line 45 to the shut-off valves. Alternatively, pressurizing medium could also be conducted away through the fluid line 36 serving as a supply line for pressurizing medium, and be utilized for actuating the shut-off valve 53 of the fluid line 36 for delivery of pressurizing medium.

In this way, the first pressure chamber 33 can be utilized as a sterilization chamber for sterilizing the section of the immersion tube 11 arranged within the pressure chamber 33, as well as the seal 29, which seals off the first pressure chamber 33 relative to the treatment chamber 8. In this way, it is assured that, in the case of axial shifting of the immersion tube 11 back into the measuring position, no germs are brought from the cylinder 9 into the treatment chamber 8. This increases, as a whole, the safety of a sterile process, in which the probe system 1 for determining measured variables of a medium contained in the process container is applied.

The above described sterilization of the immersion tube 11 and of the measuring head of the measuring probe accommodated therein within the treatment chamber 8 and the sterilization of the section of the immersion tube 11 arranged in the first pressure chamber 33 can be performed simultaneously, in order to be able to bring the measuring probe back into the process as rapidly as possible.

In addition to the above illustrated examples of embodiments, other examples of embodiments of the invention are also options. For example, the sterilization medium can also be used as pressurizing medium for the pneumatic drive for the shifting movement of the immersion tube, so that a shifting of the piston and therewith of the immersion tube and a sterilizing of the pressure chamber occurs in a single step. In this embodiment, the probe system can comprise a single fluid line opening into the first pressure chamber, through which the sterilization medium, e.g. superheated steam, is fed as pressurizing medium into the first pressure chamber, in order to displace the piston in the direction facing away from the process connection, or via which sterilization medium is discharged from the first pressure chamber, in order to shift the piston back in the direction of the process connection.

1-9. (canceled)

10. A probe system for measuring a measured variable of a measured medium, especially a measured liquid, contained in a process container, comprising:
a probe housing, which is connectable to the process container by means of a process connection;
an immersion tube axially shiftable between a measuring position, when extended out of said probe housing, and a treatment position, when retracted into said probe housing, wherein a measuring probe having a measuring head is securable in said immersion tube, wherein, in said treatment position, said measuring head is arranged within a treatment chamber formed in said probe housing;
a piston, which is arranged in an axially displaceable manner in a cylinder adjoining said treatment chamber on a side of said treatment chamber away from said process connection, and which is connected with said immersion tube; and
a fluid line serving as a supply line for a sterilization medium opens into said cylinder on a side of said piston facing said process connection.

11. The probe system as claimed in claim 10, wherein:
said piston divides said cylinder into a first pressure chamber on the side of said piston facing said process connection and a second pressure chamber on a side of said piston facing away from said process connection;
said fluid line serving as a supply line for a sterilization medium opens into said first pressure chamber; and
a fluid line serving as a supply line and/or as a discharge line for a pressurizing medium opens into said second pressure chamber.

12. The probe system as claimed in claim 10, wherein:
in addition to said fluid line serving as a supply line for a sterilization medium, another fluid line opens into said first pressure chamber, which serves as a supply line and/or as a discharge line for a pressurizing medium in said first pressure chamber.

13. The probe system as claimed in claim 10, wherein:
said immersion tube has on its front end—which is immersible in the fluid—a protective cylinder, wherein said measuring head is arranged within a perforated region of said protective cylinder.

14. The probe system as claimed in claim 10, wherein:
within said sensor housing, between said treatment chamber and said cylinder an axial guide for said immersion tube is formed, wherein said guide includes a seal, which seals said treatment chamber and said cylinder from one another.

15. The probe system as claimed in claim 12, wherein:
besides said fluid line opening into said first pressure chamber, which serves as a supply line for a sterilization medium in said first pressure chamber, and said additional fluid line opening into said first pressure chamber, which serves as a supply line and/or as a discharge line for a pressurizing medium in said first pressure chamber, the probe system includes a third fluid line opening into said first pressure chamber, which serves as a discharge line for the sterilization medium from said first pressure chamber.

16. The probe system as claimed in claim 11, wherein:
at least one said fluid lines opening into in said first pressure chamber is closable by means of a valve.

17. An operating method for a probe system for measuring a measured variable of a measured medium contained in a process container, wherein the probe system has a probe housing, which is connectable to the process container by means of a process connection, wherein the probe system is axially shiftable between a measuring position, when extended out from the probe housing, and a treatment position, when retracted into the probe housing, wherein a measuring probe having a measuring head is securable in the immersion tube, wherein, in the treatment position, the measuring head is arranged within a treatment chamber formed in the probe housing, and wherein the probe system furthermore has a piston, which is arranged in an axially displaceable manner in a cylinder adjoining the treatment chamber on a side of the treatment chamber away from the process connection, and which is connected with the immersion tube, comprising the step of:
a sterilization medium is introduced into the cylinder via a fluid line opening into the cylinder on a side of the piston facing the process connection.

18. The operating method as claimed in claim 17, wherein:
in a first step, the immersion tube is shifted into the treatment position, and in the treatment position of the immersion tube, in a second step, a sterilization medium is introduced into the treatment chamber, and a sterilization medium is introduced into the cylinder via the fluid line opening into the cylinder, and, in a third step, the immersion tube is shifted into the measuring position.
